(19) United States
(12) Patent Application Publication
(10) Pub. No.: US 2006/0141040 A1
(43) Pub. Date: Jun. 29, 2006

(54) Injectable non-aqueous suspension

(76) Inventors: Guohua Chen, Sunnyvale, CA (US); Paul R. Houston, Hayward, CA (US); Andrew Sheung-King Luk, Castro Valley, CA (US)

Correspondence Address: WOODCOCK WASHBURN LLP, ONE LIBERTY PLACE, 46TH FLOOR, PHILADELPHIA, PA, 19103, US

(21) Appl. No.: 11/305,938

(22) Filed: Dec. 19, 2005

Related U.S. Application Data
(60) Provisional application No. 60/638,448, filed on Dec. 23, 2004.

(51) Int. Cl. A61K 48/00; A61K 38/17; A61K 9/14; A61K 39/395

(52) U.S. Cl. 424/486; 514/44; 424/133.1; 514/2

(57) ABSTRACT

The present invention relates generally to compositions and methods for administering a biologically active agent, and more specifically to injectable non-aqueous suspensions.

### CROSS REFERENCE

This application claims benefit to U.S. Provisional Application Ser. No. 60/638,448, filed Dec. 23, 2004, the disclosure of which is incorporated herein by reference in its entirety.

### FIELD

The present invention relates generally to compositions and methods for administering a biologically active agent, and more specifically to injectable non-aqueous suspensions.

### BACKGROUND

Certain therapeutics, such as peptide or nucleotide based therapeutics, are generally effective only in relatively high concentrations. For example, therapies involving monoclonal antibodies (mAb) generally require the delivery of between 100 mg and 1 g of protein per dose. However, since known delivery systems are often limited to mAb concentrations up to about 50 mg/mL, such treatments commonly required administration of 2-20 mL to administer an effective amount. Typically, such large volumes must be given via intravenous infusion, which normally would need to be performed clinically. It can be readily appreciated that this is costly, inefficient, and inconvenient. Thus, it is a goal in the art to deliver these relatively large protein doses in a smaller volume, such as would be appropriate for more desirable means such as subcutaneous or intramuscular injection.

One conceptual approach would be to prepare higher concentration preparations of soluble mAbs, however, such highly concentrated solutions often result in undesirably high viscosity that renders the solution not injectable. Likewise, such highly concentrated solutions often have poor overall stability.

Another approach involves lyophilized formulations or protein crystals, but these require reconstitution prior to being delivered by injection, which makes it inconvenient. Injectable aqueous suspensions of crystallized proteins with relatively high concentration have been reported using protein crystals of insulin, but the ability to form protein crystals with other proteins has not yet demonstrated, and in fact, it is not routine.

Therefore, there is a need to develop highly concentrated protein formulations which would be injection-ready to enable delivery of a variety of therapeutic proteins with a small volume. There is a further need to develop a non-aqueous suspension vehicle having shear-thinning behavior to lower the injection force of the resulting non-aqueous suspensions. The present invention is directed to these, as well as other important ends.

### SUMMARY

The present invention describes suspension compositions, comprising a biologically active agent, and a vehicle comprising a hydrophilic viscosity enhancer and a solvent. In some embodiments, the vehicle further comprises a surfactant.

The present invention also describes methods of administering a biologically active agent, comprising suspending the biologically active agent in a vehicle comprising a hydrophilic viscosity enhancer and a solvent.

The present invention also describes methods of making an injectable formulation of biologically active agent in a concentration of at least 50 mg/mL, comprising suspending the biologically active agent in a vehicle comprising a hydrophilic viscosity enhancer and a solvent.

### BRIEF DESCRIPTION OF THE DRAWINGS

The foregoing and other objects, features and advantages of the present invention will be more readily understood upon reading the following detailed description in conjunction with the drawings as described hereinafter.

FIG. 1 is a schematic of formulated biologically active agent particles for non-aqueous suspensions.

FIG. 2 is a schematic of non-aqueous suspension vehicles.

FIG. 3 is a schematic of non-aqueous suspension formulations of biologically active agent.

FIG. 4 is a graph illustrating the viscosity as a function of PVP concentration in benzyl benzoate vehicles of the present invention.

FIG. 5 is a graph illustrating the viscosity as a function of PVP concentration in polyethylene glycol 400 vehicles of the present invention.

FIG. 6 is a graph illustrating the injection forces of non-aqueous suspensions of the present invention.

FIG. 7 is a graph illustrating the effect of sample size on in vitro release rate of lysozyme from a non-aqueous suspension formulation of the present invention (formulation 42).

FIG. 8 is a graph illustrating the in vitro release rate of BSA from the non-aqueous suspension formulations of the present invention (formulations 50, 52).

FIG. 9 is a graph illustrating identical Tryptic Peptide Mapping profile between CNTO 1275 Reference Standard Lot 4491-104 and CNTO 1275 sample from formulation 60.

FIG. 10 is a graph illustrating the Far-UV circular dichroism spectral overlay of CNTO 1275 Reference Standard Lot 4491-104, and CNTO 1275 samples from formulations 59 & 60. The data are plotted as mean residue ellipticity (deg·cm²·decimole⁻¹) versus wavelength.

FIG. 11 is a graph illustrating the physical stability (injectability) of non-aqueous suspension formulations of CNTO 1275 over shelf storage time (formulations 59, 60).

FIG. 12 is a graph illustrating the protein stability of CNTO 1275 in non-aqueous suspension formulations over shelf storage time (formulations 59, 60).

FIG. 13 is a graph illustrating subcutaneous pharmacokinetic profile of non-aqueous suspension formulation of CNTO 1275 (formulation 60) in cynomolgus monkey as compared to aqueous solution of CNTO 1275.

### DETAILED DESCRIPTION

In one embodiment, the present invention includes suspension compositions, comprising a biologically active agent, and a vehicle comprising a hydrophilic viscosity enhancer and a solvent.

In some embodiments, the vehicle further comprises a surfactant.

In one embodiment, the biologically active agent is a therapeutic agent, including small molecule, protein, antibody, mimetibody, monoclonal antibody, antibody fragment (including a diabody, triabody, or tetrabody), peptide, nucleotide, DNA, RNA, plasmid, or nucleotide fragment. In one embodiment, the biologically active agent is present in a range from 50 mg/mL to about 500 mg/mL.

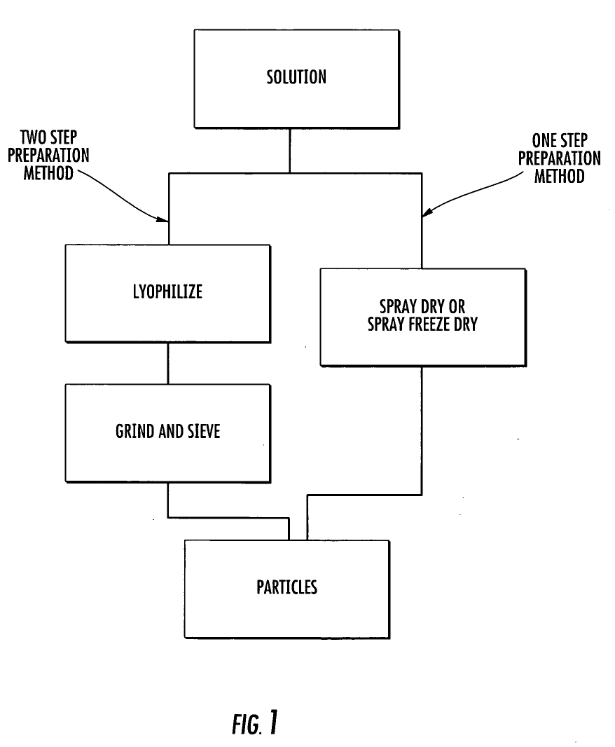

In one embodiment, the biologically active agent is formulated into a particle. Biologically active agents with particle size of about 0.1-about 250 μm with or without other excipient(s) can be produced by conventional processes such as mechanical milling or spray drying or other particle process means. Referring now to FIG. 1, there multiple paths to create biologically active agent containing particles. For example, a solution comprising biologically active agent, and in the case of proteins, a stabilizing agent and optionally buffer or pH stabilizer can be lyophilized, and then ground and sieved to particles of a desirable size. Alternatively, the solution can be spray dried or spray freeze dried to yield particles of a desirable size.

In one embodiment, the biologically active agent is present in a range from about 5 wt. % to about 60 wt. % of the composition. In one embodiment, the biologically active agent is present in a range from about 10 wt. % to about 50 wt. % of the composition.

The non-aqueous suspensions described in this invention can be applied to a variety of biological agents. Given the form of the suspension, long shelf life stability is expected. Due to the favorable shear-thinning behavior, minimal amount of viscosity enhancer is required to make the vehicles with sufficient high viscosity to support the stable suspension.

In one embodiment, the hydrophilic viscosity enhancer is polyvinylpyrrolidone, polyethylene glycol, polyproplene glycol, poly(ethylene oxide-propylene oxide-ethylene oxide), polyvinyl alcohol, poly(2-hydroxylethyl methacrylate) (PolyHEMA), poly(vinyl acetate), polyacrlamide, polyacylic acid, polyhydroxycellulose, hydroxymethylcellulose, polyesters, poly(aminoacids), polysaccharides, chitin, chitosan, hyaluronic acid, and copolymers or terpolymers thereof. In another embodiment, the hydrophilic viscosity enhancer is polyvinylpyrrolidone, polyethylene glycol, polyhydroxycellulose, polysaccharides, chitin, chitosan, or hyaluronic acid. In one embodiment, the hydrophilic viscosity enhancer is poly(vinyl pyrrolidone).

In one embodiment, the hydrophilic viscosity enhancer is present in a range from about 10 wt % to about 70 wt % of the composition. In one embodiment, the hydrophilic viscosity enhancer is present in a range from about 15 wt % to about 50 wt % of the composition.

In one embodiment, the solvent includes aromatic alcohols, lower alkyl esters of aryl acids, lower aralkyl esters of aryl acids, aryl ketones, aralkyl ketones, lower alkyl ketones, and lower alkyl esters of citric acid, and combinations thereof.

In one embodiment, the solvent is ethyl oleate, benzyl benzoate, ethyl benzoate, lauryl lactate, benzyl alcohol, lauryl alcohol, glycofurol, ethanol, tocopherol, polyethylene glycol, triacetin, a triglyceride, an alkyltriglyceride, a diglyceride, sesame oil, peanut oil, castor oil, olive oil, cottonseed oil, perfluorocarbon, N-methyl-pyrrolidone, DMSO, glycerol, oleic acid, glycofurol, lauryl lactate, perfluorocarbon, propylene carbonate, or mixtures thereof.

In one embodiment, the solvent is methyl benzoate, ethyl benzoate, n-propyl benzoate, isopropyl benzoate, butyl benzoate, isobutyl benzoate, sec-butyl benzoate, tert-butyl benzoate, isoamyl benzoate, or benzyl benzoate.

In one embodiment, the solvent is benzyl benzoate. In one embodiment, the solvent is benzyl alcohol. In one embodiment, the solvent is benzyl benzoate and benzyl alcohol.

In one embodiment, the solvent is present in a range from about 20 wt % to about 85 wt % of the composition.

In one embodiment, the vehicle further includes a surfactant. In one embodiment, the surfactant is an ionic surfactant, nonionic surfactant, or a polymeric surfactant. Examples of surfactants include ALKANOL® 189-S, ALKANOL® XC, Allyl alcohol 1,2-butoxylate-block-ethoxylate, ammonium sulfate end-capped solution, 80 wt. % in propylene glycol, 1-Decanesulfonic acid sodium salt, 98%, 4-(2,3-Dihydroxypropyl) 2-(2-methylene-4,4-dimethylpentyl)succinate potassium salt solution, 40 wt. % in water, N,N-Dimethyl-N-[3-(sulfooxy)propyl]-1-decanaminium hydroxide inner salt, N,N-Dimethyl-N-[3-(sulfooxy)propyl]-1-nonanaminium hydroxide inner salt, Dioctyl sulfosuccinate sodium salt, 96%, N-Ethyl-N-[(heptadecafluorooctyl)sulfonyl]glycine potassium salt solution, 42 wt. % in water/2-butoxyethanol, Glycolic acid ethoxylate 4-tert-butylphenyl ether, Average MN ˜380, Glycolic acid ethoxylate lauryl ether, Average MN ˜360, Glycolic acid ethoxylate lauryl ether, Average MN ˜460, Glycolic acid ethoxylate lauryl ether, Average MN ˜690, Glycolic acid ethoxylate 4-nonylphenyl ether, Average MN ˜600, Glycolic acid ethoxylate oleyl ether, Average MN ˜410, Glycolic acid ethoxylate oleyl ether, Average MN ˜540, Glycolic acid ethoxylate oleyl ether, Average MN ˜700, [3-((((Heptadecafluorooctyl)sulfonyl)amino)propy)]trimethylammonium iodide solution, 42 wt. % in 2-propanol/water, Poly(ethylene glycol) 4-nonylphenyl 3-sulfopropyl ether potassium salt, Sodium dodecylbenzenesulfonate, Technical Grade, Sodium dodecyl sulfate, 70%, Sodium dodecyl sulfate, 98%, ZONYL® 7950, ZONYL® FSA fluorosurfactant, 25 wt. % Li carboxylate salt in water: isopropanol (37.5:37.5), ZONYL® FSE fluorosurfactant, 14 wt. % in water: ethylene glycol (62:24), ZONYL® FSP fluorosurfactant, ZONYL®UR fluorosurfactant, ADOGEN® 464, ALKANOL® 6112, Allyl alcohol 1,2-butoxylate-block-ethoxylate, Allyl alcohol 1,2-butoxylate-block-ethoxylate, BRIJ®30, Average MN ˜362, BRIJ®35, Average MN ˜1,198, BRIJ®52, Average MN ˜330, BRIJ®56, Average MN ˜683, BRIJ®58, Average MN ˜1,124, BRIJ®72, Average MN 359, BRIJ®76, Average MN ˜711, BRIJ®78, Average MN ˜1,152, BRIJ®92, Average MN ˜357, BRIJ®97, Average MN 709, BRIJ®98, Average MN ˜1,150, BRIJ® 700, Average MN ˜4,670, 2,5-Dimethyl-3-hexyne-2,5-diol, 98%, Ethylenediamine tetrakis(ethoxylate-block-propoxylate) tetrol, Average MN ˜7,200, Ethylenediamine tetrakis(ethoxylate-block-propoxylate) tetrol, Average MN ˜8,000, Ethylenediamine tetrakis(propoxylate-block-ethoxylate) tetrol, Average MN ˜3,600, Ethylenediamine tetrakis(propoxylate-block-ethoxylate) tetrol, Average MN ˜15,000, IGEPAL® CA-210, Average MN ˜294, IGEPAL® CA-520, Average MN ˜427, IGEPAL® CA-720, Average MN ˜735, IGEPAL® CO-210, Average MN ˜308, IGEPAL® CO-520, IGEPAL® CO-630, Average MN ˜617, IGEPAL® CO-720, Average MN ˜749, IGEPAL® CO-890, Average MN ˜1,982, IGEPAL® CO-990, Average MN ˜4,626, IGEPAL® DM-970, MERPOL® DA surfactant, 60 wt. % in water/isobutanol (ca. 50:50), MERPOL® HCS surfactant, MERPOL® LFH surfactant, MERPOL® OJ surfactant, MERPOL® SE surfactant, MERPOL® SH surfactant, MERPOL®A surfactant, 8-Methyl-1-nonanol propoxylate-block-ethoxylate, Poly(acrylic acid) partial sodium salt, particle size 1000 μm (99%), Poly(acrylic acid) partial sodium salt solution, Average MW ˜2,000 by GPC, 60 wt. % in water, Poly[dimethylsiloxane-co-methyl(3-hydroxypropyl)siloxane]-g raft-poly(ethylene/propylene glycol), Polyethylene-block-poly(ethylene glycol), Average MN ˜1,400, Polyethylene-block-poly(ethylene glycol), Average MN ˜920, Polyethylene-block-poly(ethylene glycol), Average MN ˜875, Polyethylene-block-poly(ethylene glycol), Average MN ˜575, Poly(ethylene glycol) n-alkyl 3-sulfopropyl ether potassium salt, Poly(ethylene glycol)-block-poly(propylene glycol)-block-poly(ethylene glycol), Average MN ˜1,100, Poly(ethylene glycol)-block-poly(propylene glycol)-block-poly(ethylene glycol), Average MN ˜1,900, Poly(ethylene glycol)-block-poly(propylene glycol)-block-poly(ethylene glycol), Average MN ˜2,000, Poly(ethylene glycol)-block-poly(propylene glycol)-block-poly(ethylene glycol), Average MN ˜2,800, Poly(ethylene glycol)-block-poly(propylene glycol)-block-poly(ethylene glycol), Average MN ˜2,800, Poly(ethylene glycol)-block-poly(propylene glycol)-block-poly(ethylene glycol), Average MN ˜2,900, Poly(ethylene glycol)-block-poly(propylene glycol)-block-poly(ethylene glycol), Average MN ˜4,400, Poly(ethylene glycol)-block-poly(propylene glycol)-block-poly(ethylene glycol), Average MN ˜5,800, Poly(ethylene glycol)-block-poly(propylene glycol)-block-poly(ethylene glycol), Average MN ˜8,400, Poly(ethylene glycol) 2-[ethyl[(heptadecafluorooctyl)sulfonyl]amino]ethyl ether, Poly(ethylene glycol) 2-[ethyl[(heptadecafluorooctyl)sulfonyl]amino]ethyl methyl ether, Poly(ethylene glycol) myristyl tallow ether, Average MN ˜3,000, Poly(hexafluoropropylene oxide) monocarboxylic acid, chloro terminated, Average MN ˜500, Polyoxyethylene sorbitan tetraoleate, Polyoxyethylene sorbitol hexaoleate, Polyoxyethylene(6) tridecyl ether, Mixture of C11 to C14 iso-alkyl ethers with C13 iso-alkyl predominating, Polyoxyethylene(12) tridecyl ether, Mixture of C11 to C14 iso-alkyl ethers with C13 iso-alkyl predominating, Polyoxyethylene(18) tridecyl ether, Mixture of C11 to C14 iso-alkyl ethers with C13 iso-alkyl predominating, Poly(propylene glycol)-block-poly(ethylene glycol)-block-poly(propylene glycol), Average MN ˜2,000, Poly(propylene glycol)-block-poly(ethylene glycol)-block-poly(propylene glycol), Average MN ˜2,700, Poly(propylene glycol)-block-poly(ethylene glycol)-block-poly(propylene glycol), Average MN ˜3,300, Sorbitan monolaurate, Sorbitan monooleate, Sorbitan monopalmitate, Sorbitan monostearate, Sorbitan sesquioleate, Sorbitan trioleate, TERGITOL® NP-9, 2,4,7,9-Tetramethyl-5-decyne-4,7-diol ethoxylate, Average MN ˜380, Average MW ˜395, 2,4,7,9-Tetramethyl-5-decyne-4,7-diol ethoxylate, Average MN ˜670, Average MW ˜700, 2,4,7,9-Tetramethyl-5-decyne-4,7-diol ethoxylate, Average MN ˜1,200, Average MW ˜1,250, 2,4,7,9-Tetramethyl-5-decyne-4,7-diol, mixture of (±) and meso, 98%, TRITON® X-100, TRITON® X-100, reduced, TRITON® N-101, reduced, TRITON® X-114, TRITON® X-114, reduced, 99+%, TRITON® X-114, reduced, TRITON® X-405, reduced, TRITON® X-405 solution, 70 wt. % in water, TRITON® SP-135, TRITON® SP-190, TWEEN® 20, Average MN ˜1,228, TWEEN®20 solution, 72 wt. % in water, TWEEN® 40, Average MN ˜1,284, TWEEN® 60, Average MN ˜1,312, TWEEN® 80, Average MN ˜1,310, TWEEN® 85, Average MN ˜1,839, PLURONIC® F68, PLURONIC® F127, PLURONIC® L61, PLURONIC® L81, PLURONIC® L92, PLURONIC® L121 etc, TWEEN 20, TWEEN 80, CREMOPHOR® EL 35, CREMOPHOR® EL 40, CREMOPHOR® EL 60, ZONYL® FSN, ZONYL® FSN-100, ZONYL® FSO, and ZONYL® FSO-100.

In one embodiment, the surfactant is a polyoxyethylene sorbitan-containing composition or a block copolymer of propylene oxide and ethylene oxide, a block copolymer derived from the addition of ethylene oxide and propylene oxide to ethylenediamine, polyethelene glycol, or polyethylene oxide. In one embodiment, the surfactant is TWEEN 20 (polyoxyethylene sorbitan monolaureate) or TWEEN 80 (polyoxyethylene sorbitan monooleat).

In one embodiment, the surfactant is a block copolymer of propylene oxide and ethylene oxide is of a formula HO-(ethylene oxide)ₓ-(propylene oxide)_{y}-(ethylene oxide)x′—H. In one embodiment, x is in a range from about 2 to about 150, y is in a range from about 20 to about 70, and x′ is in a range from about 2 to about 150. In one embodiment, the surfactant is PLURONIC F68 surfactant.

In one embodiment, the surfactant is present in a range from about 0.1 wt % to about 5 wt % of the composition.

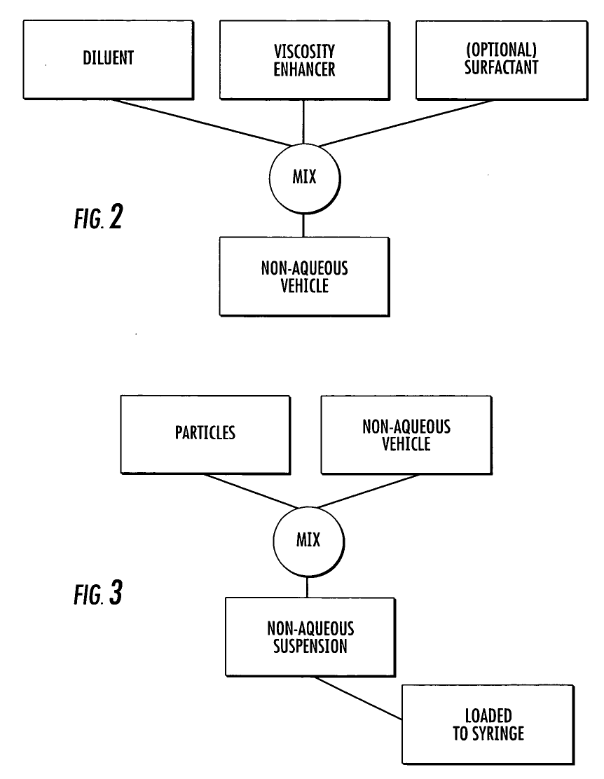

As show in FIG. 2, the viscosity enhancer, diluent (solvent above), and optionally, surfactant, can be mixed to form the non-aqueous vehicle.

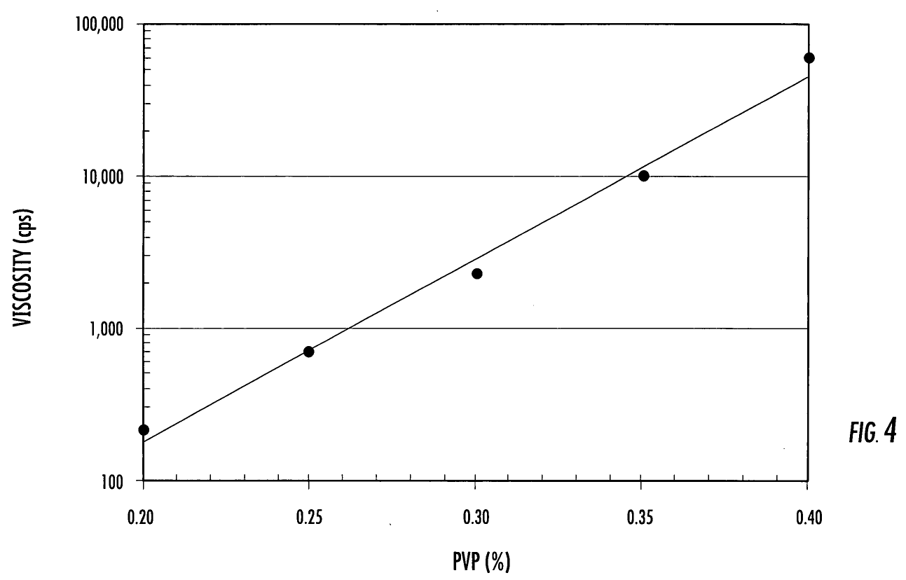

Turning to FIG. 3, in one embodiment, the biologically active agent containing particles and non-aqueous vehicle are combined to form a non-aqueous suspension.

The non-aqueous suspensions are prepared by mixing the biologically active agent into the non-aqueous polymer solution (vehicle) with the biologically active agent loading of about 10-50 percent by weight.

The present non-aqueous suspensions attain very high protein loading (about 50 mg/mL or greater, preferably about 100 mg/mL or greater). This would not be possible in an aqueous formulation without loss of injectability and/or stability. In one embodiment, the suspension is pre-loaded in a syringe and thus is injection ready with no mixing or reconstitution. The formulation can be administrated subcutaneously or intramuscularly. In one embodiment, the suspension vehicles utilizes hydrophilic polymers as viscosity enhancers. The protein is kept in its solid form, thus, long shelf life stability is expected.

In one embodiment, the present invention includes a pharmaceutical composition, comprising the above-described suspension composition and a pharmaceutically acceptable excipient. Examples of excipients include all known excipients, include sugars, pH modifiers, reducing agents, and antioxidants. Embodiments of the present invention may use a single excipient or a combination of excipients.

Sugar excipients include sucrose, trehalose, and the like.

pH modifying excipients include inorganic salts, such as zinc carbonate, magnesium carbonate, calcium carbonate, magnesium hydroxide, calcium hydrogen phosphate, calcium acetate, calcium hydroxide, calcium lactate, calcium maleate, calcium oleate, calcium oxalate, calcium phosphate, magnesium acetate, magnesium hydrogen phosphate, magnesium phosphate, magnesium lactate, magnesium maleate, magnesium oleate, magnesium oxalate, zinc acetate, zinc hydrogen phosphate, zinc phosphate, zinc lactate, zinc maleate, zinc oleate, zinc oxalate, and combinations thereof.

Reducing agent excipients include cysteine or methionine.

Antioxidant excipients include d-alpha tocopherol acetate, dl-alpha tocopherol, ascorbyl palmitate, butylated hydroxyanidole, ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propylhydroxybenzoate, trihydroxybutylrophenone, dimethylphenol, diterlbulylphenol, vitamin E, lecithin, ethanolamine, and combinations thereof.

Methods of making the composition include: 1) premixing the excipient with the beneficial agent before mixing into the vehicle, 2) premixing the excipient with the vehicle before mixing in the beneficial agent, or 3) loading the excipient and the beneficial agent separately into the vehicle.

In one embodiment, the pharmaceutical composition further comprises a buffer. Buffers include all known buffers, including citrate, succinate, cold phosphate buffered saline (PBS), etc.

In one embodiment, the pharmaceutical composition is an immediate release formulation.

In one embodiment, the pharmaceutical composition is substantially all released within 24 hours.

In one embodiment, the pharmaceutical composition is fluidly injectable at 25° C.

In one embodiment, the pharmaceutical composition is administered subcutaneously or intramuscularly.

In one embodiment, the present invention includes a dosage kit comprising the above-described suspension composition and a syringe. In one embodiment, the syringe is an auto-injector syringe. In one embodiment, the syringe is divided such that the biologically active agent and the vehicle are separate until being mixed before injection. In one embodiment, two syringes are provided in the kit, the biologically active agent being stored in the first syringe and the vehicle being stored in the second syringe being mixed before injection.

In one embodiment, the kit is adapted to be self-administered by a patient in need thereof.

In yet another embodiment of the present invention, a vehicle is provided for combining with a biologically active agent to form a suspension composition, the vehicle comprising a hydrophilic viscosity enhancer and a solvent, and an optional surfactant, all as described above.

In yet another embodiment of the present invention, a method of administering a biologically active agent is provided, the method comprising suspending the biologically active agent in the previously described vehicle composition, and injecting the resulting composition into a patient in need thereof. In one embodiment, the biologically active agent is a monoclonal antibody.

In yet another embodiment of the present invention, a method of making an injectable formulation of biologically active agent in a concentration of at least 50 mg/mL is provided, the method comprising suspending the biologically active agent in the above described vehicle composition.

The present compositions are further described in the following examples.

### EXAMPLES

### Example 1

Particle Preparation of Biologically Active Agent by Lyophilization Methods

Lysozyme (Sigma, St. Louis, Mo., USA) is dissolved in 6.5 mM sodium phosphate buffer, pH 6.0 with a protein concentration of 65 mg/mL. To this solution, sucrose (Sigma, St. Louis, Mo., USA) and a surfactant, such as TWEEN 80 or polysorbate 80, are added with the concentration of sucrose and TWEEN 80 in the final solution of 5.5% and 0.0065% w/v, respectively. This solution is lyophilized following the conditions in TABLE 1.

TABLE 1

| | | Chamber | Hold Time | |
|---|---|---|---|---|
| Process Step | Shelf Temperature(° C.) | Pressure (mBar) | (hour) | |
| Loading | +5° C. | N/A | 2 | |
| Freezing | −50° C. (rate 0.5° C.) | N/A | 2 | |
| Freezing | −50° C. | N/A | 2.5 | |
| Vacuum on | −50° C. | 120 mT | 0.5 | |
| Vacuum | −50° C. | 120 mT | 0.5 | |
| hold | | | | |
| 1° Drying | −10° C. (rate 1° C./min) | 120 mT | 0.75 | |
| 1° Drying | −10° C. | 120 mT | 24 | |
| 2° Drying | 0° C. (rate 0.1° C./min) | 80 mT | 1.7 | |
| 2° Drying | 0° C. | 80 mT | 2 | |
| 2° Drying | +35° C. (rate 0.25° C./min) | 80 mT | 2.3 | |
| 2° Drying | +35° C. | 80 mT | 10 | |
| 2° Drying | +20° C. (rate 1° C./min) | 80 mT | 0.25 | |
| 2° Drying | +20° C. | 80 mT | 2 | |
| | | Min. Total time = | 50.5 | h |

The lysozyme particles with controllable particle size range are prepared by grinding the above described lyophilized formulation with a Waring blender and sieving through a series of sieve with determined mesh sizes. Particles with sizes of <about 38 μm, between about 38-about 63 μm, <about 125 μm, or <about 250 μm etc. are produced this way (FIG. 1).

In the similar ways to those described above, particles of bovine serum albumin (BSA, Sigma, St. Louis, Mo., USA) are prepared. Likewise, particles of a monoclonal antibody, for example, CNTO 1275 human mAb to anti-IL-12p40, CNTO 148 muman anti-TNFα, etc. from Centocor Inc. USA, can be prepared as described above (details of example formulations are summarized in TABLE 2).

TABLE 2

| | | Biological | | |
|---|---|---|---|---|
| | Biological | active agent | Sucrose | TWEEN 80 |
| Formulation | active agent | (mg/mL) | (% w/v) | (% w/v) |
| 1 | Lysozyme | 65 | 5.5 | 0.0065 |
| 2 | Lysozyme | 65 | 3.0 | 0.0065 |
| 3 | Lysozyme | 100 | 4.5 | 0.0065 |
| 4 | BSA | 65 | 5.5 | 0.0065 |
| 5 | BSA | 65 | 3.0 | 0.0065 |
| 6 | BSA | 100 | 4.5 | 0.0065 |
| 7 | CNTO 1275 | 65 | 5.5 | 0.0065 |
| 8 | CNTO 148 | 65 | 5.5 | 0.0065 |

### Example 2

Particle Preparation of Biologically Active Agent by Spray Drying Methods

Similarly, the solution of lysozyme or BSA formulated as described in Example 1 can be diluted spray dried (FIG. 1). The solution may be diluted to ca. 20 mg/mL with DI water in some case. The spray-dried particles were produced using a Yamato Mini Spray dryer set at the following parameters in TABLE 3:

TABLE 3

| Spray Dryer Parameter | Setting |
|---|---|
| Atomizing Air | 2 psi |
| Inlet Temperature | 120° C. |
| Aspirator Dial | 7.5 |
| Solution Pump | 2-4 |
| Main Air Valve | 40-45 psi |

The particles having a size range between 1-10 microns were obtained (details of example formulations are summarized in TABLE 4).

TABLE 4

| | | Biological | | |
|---|---|---|---|---|
| | Biological | active agent | Sucrose | TWEEN 80 |
| Formulation | active agent | (mg/mL) | (% w/v) | (% w/v) |
| 9 | Lysozyme | 65 | 5.5 | 0.0065 |
| 10 | Lysozyme | 20 | 1.7 | 0.0020 |
| 11 | Lysozyme | 65 | 3.0 | 0.0065 |
| 12 | BSA | 65 | 5.5 | 0.0065 |
| 13 | BSA | 20 | 1.7 | 0.0020 |
| 14 | BSA | 65 | 3.0 | 0.0065 |

### Example 3

Non-Aqueous Suspension Vehicle Preparation

Polyvinyl pyrrolidone (PVP), a hydrophilic polymer, (Povidone, USP KOLLIDONE 17PF, BASF), is dissolved in benzyl benzoate (BB) with polymer concentration of 20-70% by weight. A surfactant, PLURONIC® F68 or POLOXAMER® 188, from BASF, is added into this solution with an amount about 0.1-8% by weight of PVP/BB solution (FIG. 2).

Similarly, a vehicle formulation of PVP/BB with polymer concentration of 20-70% by weight can be prepared with a surfactant of TWEEN 80, or polysorbate 80 in an amount of 0.1-4% by weight of PVP/BB solution (details of example formulations are summarized in TABLE 5).

TABLE 5

| | | | Pluronic F68 | |
|---|---|---|---|---|
| | PVP | BB | in PVP/BB | TWEEN 80 in PVP/BB |
| Formulation | (wt %) | (wt %) | (% w/v) | (% w/v) |
| 15 | 20 | 80 | 1 | 0 |
| 16 | 30 | 70 | 1 | 0 |
| 17 | 25 | 75 | 2 | 0 |
| 18 | 30 | 70 | 2 | 0 |
| 19 | 50 | 50 | 1 | 0 |
| 20 | 30 | 70 | 4 | 0 |
| 21 | 20 | 80 | 0 | 1 |
| 22 | 30 | 70 | 0 | 1 |
| 23 | 30 | 70 | 0 | 4 |
| 24 | 50 | 50 | 0 | 1 |
| 25 | 30 | 70 | 0 | 2 |
| 26 | 30 | 70 | 1 | 1 |

### Example 4

Compatibility of Biologically Active Agent with Non-Aqueous Suspension Vehicles

The compatibility of biologically active agent such as monoclonal antibodies in various solvents or oils as well as representative suspension vehicles of this invention is tested. Two lyophilized preparation of Mabs, CNTO 1275 and CNTO 148 were evaluated (Formulations 7 & 8 in TABLE 2).

In order to analyze the mAB from the mixture with vehicles, the mAB is extracted from the mixture using the following extraction procedures: an excess of the pre-chilled extraction solvent (mixture of dichloromethane/acetone, 1:1) is added to each sample. After mixing, the sample is centrifuged and the supernatant removed. The remaining pellet is then washed twice with the pre-chilled extraction solvent and dried through speed-vac. The sample is reconstituted is PBS buffer, pH 6.5 and analyzed for monomer content with SEC-HPLC.

Tables 6 & 7 summarize the stability of lyophilized CNTO 1275 and CNTO 148 suspended in different solvent/vehicles of present invention after incubation at 37° C. for up to 8 days. Except for the suspensions comprising benzyl alcohol (BA), both CNTO 1275 and CNTO 148 were found stable with no noticeable loss in protein monomer content (as measured by SEC-HPLC) in suspension solvents, oils, vehicles investigated, after incubation at 37° C. for up to 8 days.

TABLE 6

| | | Monomer | |
|---|---|---|---|
| | Solvent, or oil | at day 1 | Monomer at day 8 |
| Formulation* | or vehicles | (%) | (%) |
| 28 | BB | 99.4 ± 0.0 | 99.2 ± 0.1 |
| 29 | BB/BA, 75/25 | 95.9 ± 1.6 | 83.6 ± 0.5 |
| 30 | Sesame oil | 99.5 ± 0.0 | 99.4 ± 0.0 |
| 31 | perfluorodecalin | 99.4 ± 0.0 | 99.1 ± 0.0 |
| 32 | Ethyl oleate | 99.5 ± 0.0 | 99.1 ± 0.1 |
| Control^{a} | Proceeded | 99.4 ± 0.0 | 98.5 ± 0.2 |
| | with extraction | | |

*ca. 10 mg of formulation 7 (TABLE 2) immersed in ca. 0.5 mL of solvent, oil or vehicles, incubated at 37° C. for up to 8 days;
^{a}Formulation 7 particles without immersed in solvent, or oil or vehicles, but incubated at 37° C. for up to 8 days and proceeded with solvent extraction as with the formulations 28-32.

TABLE 7

| | | Monomer at day 8 |
|---|---|---|
| Formulation* | Solvent, or oil or vehicles | (%) |
| 33 | BB | 96.9 ± 0.1 |
| 34 | BB/BA, 75/25 | 90.8 ± 1.4 |
| 35 | PVP/BB, 30/70 | 97.2 ± 0.1 |
| 36 | Sesame oil | 97.0 ± 0.1 |
| 37 | perfluorodecalin | 96.7 ± 0.3 |
| 38 | Ethyl oleate | 97.1 ± 0.0 |
| Control^{a} | Proceeded with extraction | 96.6 ± 0.3 |
| Control^{b} | No extraction | 96.6 ± 0.1 |
| Control^{c} | Proceeded with extraction | 97.5 ± 0.0 |
| Control^{d} | No extraction | 97.5 ± 0.1 |

*ca. 10 mg of formulation 7 (TABLE 2) immersed in ca. 0.1 mL of solvent, oil or vehicles, incubated at 37° C. for up to 8 days;
^{a}Formulation 8 particles without immersed in solvent, or oil or vehicles, but incubated at 37° C. for up to 8 days and proceeded with solvent extraction as with the formulations 33-38;
^{b}Formulation 8 particles without immersed in solvent, or oil or vehicles, incubated at 37° C. for up to 8 days but not proceeded with solvent extraction as with the formulations 33-38;
^{c}Formulation 8 particles without immersed in solvent, or oil or vehicles, and without incubation at 37° C. for up to 8 days but proceeded with solvent extraction as with the formulations 33-38;
^{d}Formulation 8 particles without immersed in solvent, or oil or vehicles, and without incubation at 37° C. for up to 8 days and without solvent extraction.

### Example 5

Preparation of Non-Aqueous Suspension with Biologically Active Agent

Biologically active agent particles, such as ones prepared in examples 1 & 2 above, are mixed with the non-aqueous suspension vehicles such as PVP/BB/Pluronic F68 or PVP/BB/TWEEN 80 as described in the Example 3 above using an overhead mixer. Mixing is performed at room temperature inside a dry box. The particles and vehicles are first weighed and transferred into a 25 cc glass syringes. The particle loading is about 10-50% by weight leading to the protein concentration in the final formulation about 50-500 mg/mL. An electric stirrer with a stainless steel spatula blade is used to blend the particles into the vehicles at 50-300 rpm for 5 minutes. The suspension formulation is filled into a glass injection syringes, yielding a syringe-ready dosage form (FIG. 3). The formulations are stored at refrigerated temperature prior to injection (details of example formulations are summarized in TABLE 8).

TABLE 8

| | | Drug | | |
|---|---|---|---|---|
| | Drug Particle | particles | Vehicle | Vehicles |
| Formulation | formulations | (wt %) | formulations | (wt %) |
| 39 | Formulation 1 | 20 | Formulation 17 | 80 |
| 40 | Formulation 1 | 40 | Formulation 17 | 60 |
| 41 | Formulation 1 | 30 | Formulation 18 | 70 |
| 42 | Formulation 1 | 40 | Formulation 18 | 60 |
| 43 | Formulation 2 | 40 | Formulation 18 | 60 |
| 44 | Formulation 3 | 40 | Formulation 18 | 60 |
| 45 | Formulation 1 | 40 | Formulation 22 | 60 |
| 46 | Formulation 1 | 40 | Formulation 23 | 60 |
| 47 | Formulation 9 | 40 | Formulation 17 | 60 |
| 48 | Formulation 9 | 40 | Formulation 18 | 60 |
| 49 | Formulation 4 | 20 | Formulation 17 | 80 |
| 50 | Formulation 4 | 40 | Formulation 17 | 60 |
| 51 | Formulation 4 | 30 | Formulation 18 | 70 |
| 52 | Formulation 4 | 40 | Formulation 18 | 60 |
| 53 | Formulation 5 | 40 | Formulation 18 | 60 |
| 54 | Formulation 6 | 40 | Formulation 18 | 60 |
| 55 | Formulation 12 | 40 | Formulation 17 | 60 |
| 56 | Formulation 12 | 40 | Formulation 18 | 60 |
| 57 | Formulation 4 | 40 | Formulation 22 | 60 |
| 58 | Formulation 4 | 40 | Formulation 23 | 60 |
| 59 | Formulation 7 | 40 | Formulation 17 | 60 |
| 60 | Formulation 7 | 40 | Formulation 18 | 60 |

### Example 6

Viscosity Measurements on Depot Gel Vehicles

Viscosity of the non-aqueous suspension vehicles formulated as described in Example 3 above was tested using a Bohlin CVO 120 rheometer. All testing were done at 24° C. using 20 mm parallel plates.

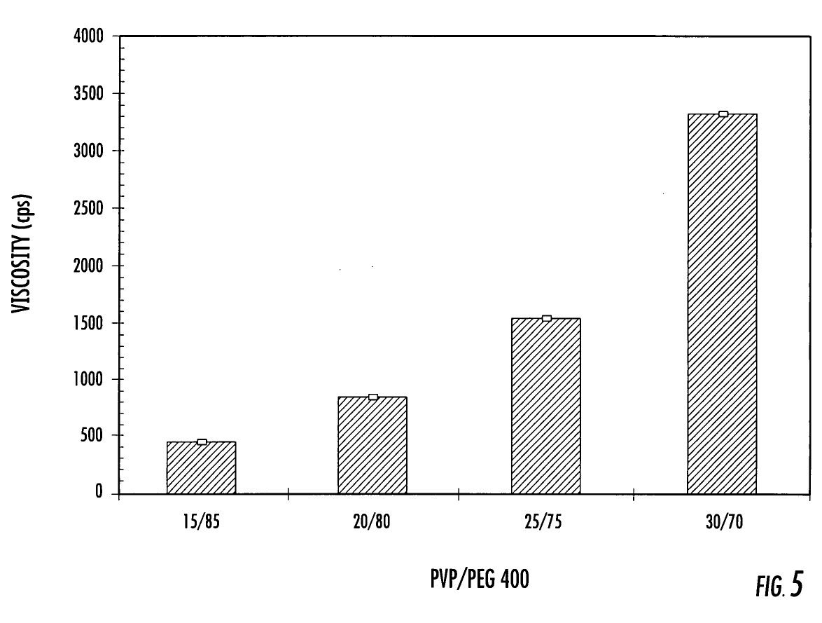

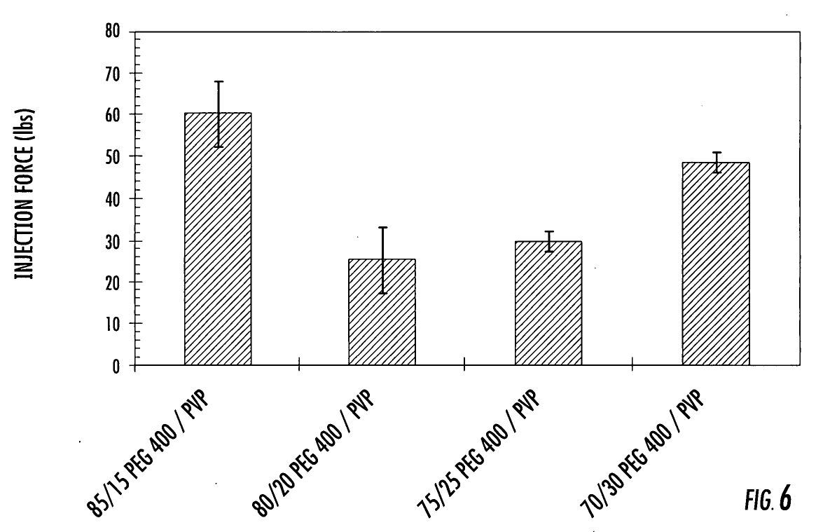

FIGS. 4 & 5 illustrated the viscosity of non-aqueous vehicle formulations using either BB (FIG. 4) or polyethylene glycol 400 (PEG 400) (FIG. 5) as solvent as a function of PVP content in the vehicle formulations. The higher the PVP content in the vehicle, the higher the viscosity. A certain viscosity of the vehicles might be desirable in order to prepare stable suspensions. As demonstrated in FIGS. 4 & 5, the viscosity of the vehicles can be tuned by the polymer concentration to meet a desirable viscosity.

### Example 7

Injectability Test of Non-Aqueous Suspensions with Biologically Active Agent

Injectability of the non-aqueous suspension is evaluated by measuring the force required to push whole content of the suspension formulations in the syringe through a fine gauge needle. The suspension formulations are loaded in the Hamilton 500 ul GASTIGHT® syringe. The injection force of the non-aqueous suspension formulations was tested on an Instron tensile testing instrument, where the maximum force required to move the syringe plunger was determined. Prior to injection force testing, all samples will be equilibrated at room temperature (for ca. 1-2 hours), for the samples when stored at 4° C. The injection rate is set to be 1 cc/min or a crosshead speed using 21 G 1″ needle.

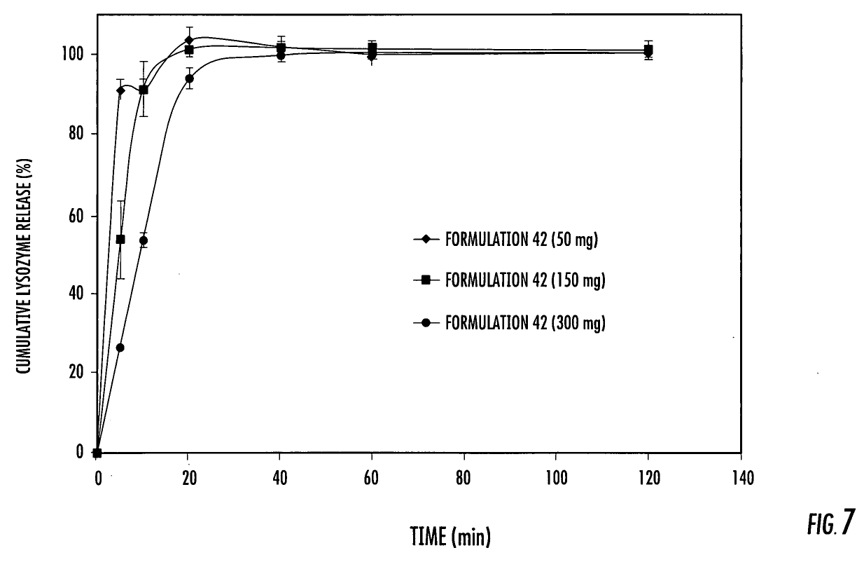

FIG. 6 illustrates the forces required to push the suspension formulations (loaded with 40 wt % BSA particles, formulation 4) as a function of PVP concentration in the vehicle. In general, the higher the PVP concentration in the vehicles, the higher the force required to push the formulations out of syringe through a fine needle, except for the vehicles with very low PVP concentrations in which large variability may be experienced.

### Example 8

In Vitro Release Rate of Biologically Active Agent from Non-Aqueous Suspensions

The in vitro release of biologically active agent from the non-aqueous suspension formulations is conducted in 50 mM PBS pH 7.4 at 37° C. A certain amount of suspension formulation is placed in a 3 mL vacutainer, to which ca. 2 mL of PBS buffer is added. Load the Vacutainer on an auto rotator and place system inside a 37° C. oven. At the predetermined time points, 0.5 mL of supernatant is withdrawn and replaced with 0.5 mL fresh PBS buffer. The withdrawn supernatant is analyzed by SEC for the active.

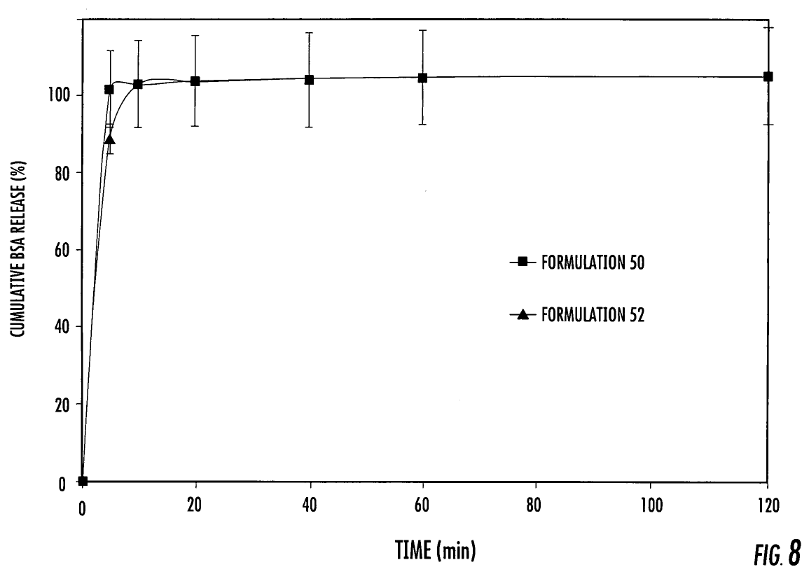

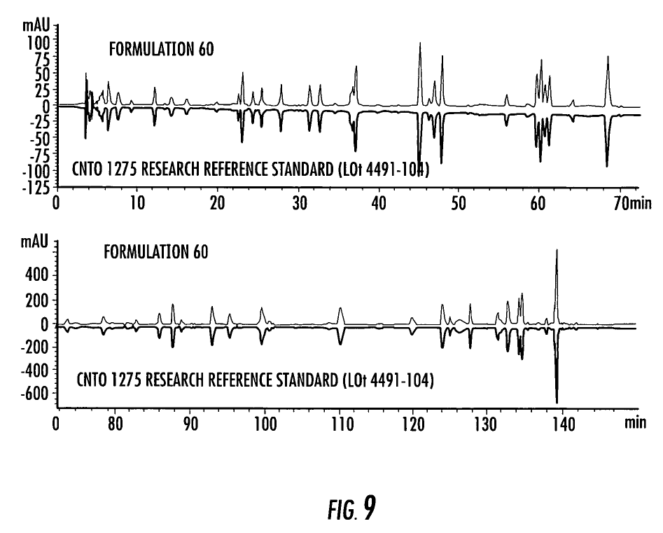

FIGS. 7 & 8 illustrate the cumulative release of active (lysozyme, FIG. 7; BSA, FIG. 8) from the non-aqueous suspension formulations. Immediate release of active from the non-aqueous suspension formulations of the present invention is achieved.

### Example 9

Characterization of Monoclonal Antibody After Suspended in Non-Aqueous Formulations

A monoclonal antibody such as CNTO 1275 is suspended in non-aqueous formulations (Formulations 59 & 60 in TABLE 8 of Example 5). The CNTO 1275 is extracted from the non-aqueous suspension formulations following the procedures described in Example 4 above. The extracted CNTO 1275 from the non-aqueous suspension formulation is characterized with a battery of comparative analytical methods (see TABLE 9 below).

TABLE 9

| Comparative Analytical Methods | Results |
|---|---|
| Analytical Biochemical Tests | |
| SEC-HPLC | Pass |
| Tryptic Peptide Mapping | Pass |
| UV, pH, OD (concentration) | Pass |
| SDS-PAGE | Pass |
| Isoelectric Focusing (IEF) | Pass |
| Analytic Biophysical Tests | |
| Circular Dichroism (CD) | Pass |
| Sedimentation velocity ultracentrifugation | Pass |
| (SV-AUC) | |
| Differential Scanning Calorimetry (DSC) | Pass |
| Analytical; Bioactive Test | |
| Bioactivity assay | Pass |

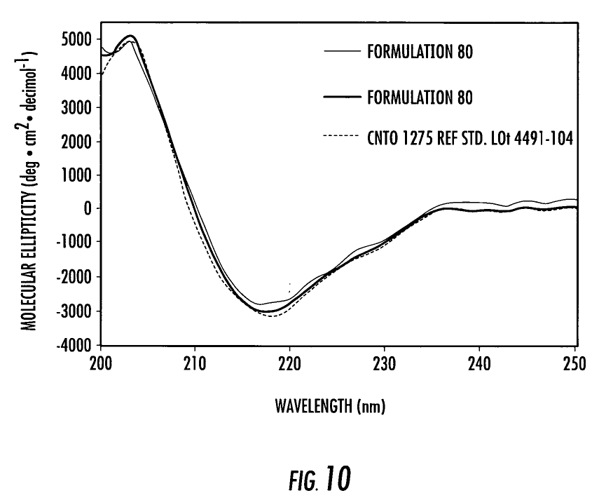

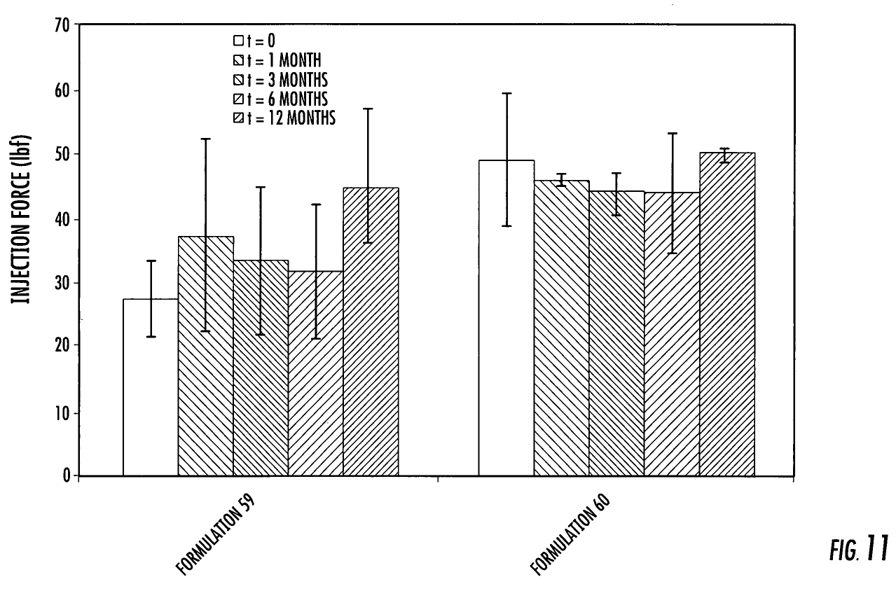

Selected results from the comparative analysis are exhibited in FIG. 9, FIG. 10, and TABLE 10. As compared to CNTO 1275 standard, the CNTO 1275 protein extracted from the non-aqueous suspension formulations showed identical primary, secondary and tertiary structures as the lyophilized control suggesting that Mab integrity is stable in the non-aqueous suspension vehicles. TABLE 10 summarizes the sedimentation coefficients for CNTO 1275 samples from formulations 59 & 60 and CNTO 1275 Reference Standard Lot 4491-104.

TABLE 10

| | S⁰_{20,w} |
|---|---|
| Formulations | (Svedberg) |
| CNTO 1275 standard (Lot 4491-104) | 5.6 |
| CNTO 1275 in Formulation 59 | 5.7 |
| CNTO 1275 in Formulation 60 | 5.7 |

### Example 10

Stability of Biologically Active Agent in Non-Aqueous Suspensions

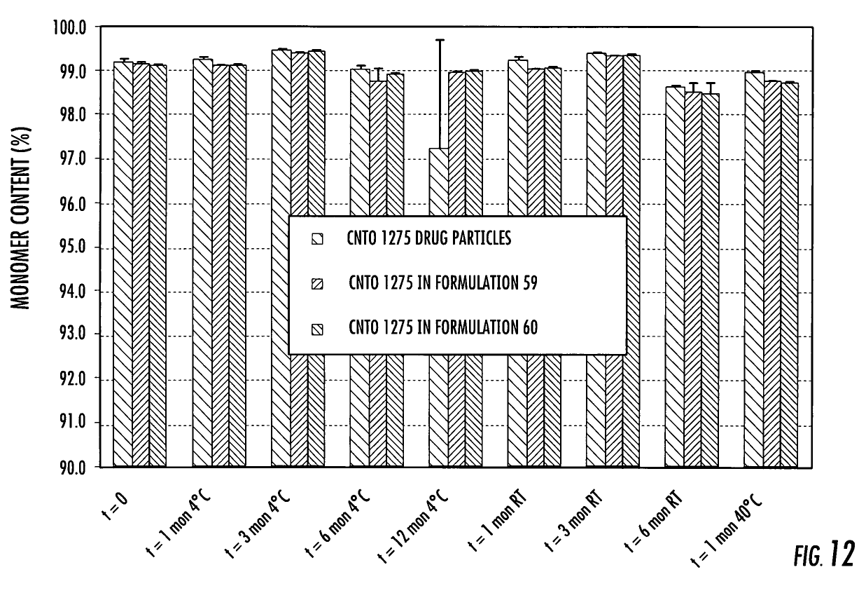

FIG. 11 demonstrates the protein stability of CNTO 1275 in the non-aqueous suspension formulation after storage at three different temperatures. The stability was evaluated by the monomer content as determined by SEC-HPLC. There are no significant changes in monomer content of CNTO 1275 in the representative suspension formulations evaluated after storage at 37° C. for 1 month, room temperature for 6 months, and refrigerated temperature for 12 months, respectively.

### Example 11

Physical Stability of Non-Aqueous Suspensions

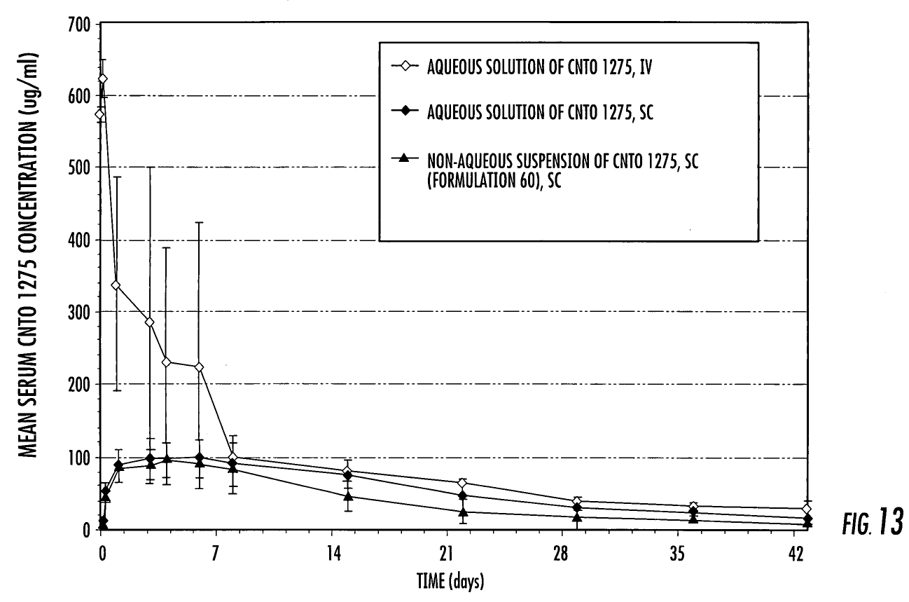

FIG. 12 illustrates the physical stability of various suspension formulations upon storage, determined by the change in force required to inject the full content of suspension through a 21 G needle (injectability) at room temperature. It can be seen that there are essentially no significant changes on the suspension formulations after storage for up to 12 months at refrigerated temperature, indicating that the suspension formulations are physically stable under the investigated storage temperature.

### Example 12

Pharmacokinetics of Biological Active Agent from the Non-Aqueous Suspensions

An in vivo PK study was performed with the representative non-aqueous suspension formulation (Formulation 60) of CNTO 1275 in cynomolgus monkey by subcutaneous (SC) injection with target dose of 10 mg CNTO 1275/kg. The SC injection of aqueous solution of CNTO 1275 was tested as control and an IV injection of aqueous solution of CNTO 1275 was also tested in order to calculate the absolute bioavailability (BA). FIG. 13 below illustrates the PK profiles of the non-aqueous suspension formulation as well as the aqueous solution control. The representative non-aqueous suspension formulation of CNTO 1275 showed essentially similar PK profile to that of the aqueous solution control, with very similar maximum concentration (Cₘₐₓ), time to reach the Cₘₐₓ (Tₘₐₓ), as well as bioavailability (BA) (see TABLE 11).

TABLE 11

| | Admin- | | | |
|---|---|---|---|---|
| | istration | Tₘₐₓ | Cₘₐₓ | BA |
| Formulation | route | (days) | (μg/mL) | (%) |
| Aqueous solution of | I.V. | 0.19 ± 0.10 | 746.6 ± 212.7 | |
| CNTO 1275 | | | | |
| Aqueous solution of | SC | 2.67 ± 2.08 | 99.6 ± 26.8 | 58 ± 14 |
| CNTO 1275 | | | | |
| Non-aqueous | SC | 1.67 ± 1.15 | 98.7 ± 31.1 | 37 ± 18 |
| solution of | | | | |
| CNTO 1275 | | | | |
| (Formulation 60) | | | | |

The disclosures of each patent, patent application, and publication cited or described in this document are hereby incorporated herein by reference, in their entireties.

Various modifications of the invention, in addition to those described herein, will be apparent to those skilled in the art from the foregoing description. Such modifications are also intended to fall within the scope of the appended claims.

What is claimed:

1. A suspension composition, comprising:
biologically active agent; and
a vehicle comprising a hydrophilic viscosity enhancer and a solvent.

2. The composition of claim 1, wherein the biologically active agent is a therapeutic agent.

3. The composition of claim 2, wherein the therapeutic agent is a small molecule, protein, peptide, nucleotide, DNA, RNA, plasmid, nucleotide fragment, antibody, monoclonal antibody, mimetibody, antibody fragment, diabody, triabody, or tetrabody.

4. The composition of claim 1, wherein the biologically active agent is present in a range from 50 mg/mL to about 500 mg/mL.

5. The composition of claim 1, wherein the biologically active agent is present in a range from about 5 wt. % to about 60 wt. % of the composition.

6. The composition of claim 1, wherein the biologically active agent is present in a range from about 10 wt. % to about 50 wt. % of the composition.

7. The composition of claim 1, wherein the hydrophilic viscosity enhancer is polyvinylpyrrolidone, polyethylene glycol, polyproplene glycol, poly(ethylene oxide-propylene oxide-ethylene oxide), polyvinyl alcohol, poly(2-hydroxylethyl methacrylate) (PolyHEMA), poly(vinyl acetate), polyacrlamide, polyacylic acid, polyhydroxycellulose, hydroxymethylcellulose, polyesters, poly(aminoacids), polysaccharides, chitin, chitosan, hyaluronic acid, and copolymers or terpolymers thereof.

8. The composition of claim 1, wherein the hydrophilic viscosity enhancer is poly(vinyl pyrrolidone).

9. The composition of claim 1, wherein the hydrophilic viscosity enhancer is present in a range from about 10 wt % to about 70 wt % of the composition.

10. The composition of claim 1, wherein the hydrophilic viscosity enhancer is present in a range from about 15 wt % to about 50 wt % of the composition.

11. The composition of claim 1, wherein the solvent is aromatic alcohol, lower alkyl ester of aryl acid, lower aralkyl ester of aryl acid, aryl ketone, aralkyl ketone, lower alkyl ketone, lower alkyl ester of citric acid, ethyl oleate, benzyl benzoate, methyl benzoate, ethyl benzoate, n-propyl benzoate, isopropyl benzoate, butyl benzoate, isobutyl benzoate, sec-butyl benzoate, tert-butyl benzoate, isoamyl benzoate, lauryl lactate, benzyl alcohol, lauryl alcohol, glycofurol, ethanol, tocopherol, polyethylene glycol, triacetin, a triglyceride, an alkyltriglyceride, a diglyceride, sesame oil, peanut oil, castor oil, olive oil, cottonseed oil, perfluorocarbon, N-methyl-pyrrolidone, DMSO, glycerol, oleic acid, glycofurol, lauryl lactate, perfluorocarbon, propylene carbonate, or mixtures thereof.

12. The composition of claim 1, wherein the solvent is benzyl benzoate, benzyl alcohol, or benzyl benzoate and benzyl alcohol.

13. The composition of claim 1, wherein the solvent is present in a range from about 20 wt % to about 85 wt % of the composition.

14. The composition of claim 1, further comprising an ionic surfactant, nonionic surfactant, or a polymeric surfactant.

15. The composition of claim 14, wherein the surfactant is a polyoxyethylene sorbitan-containing composition, a block copolymer of propylene oxide and ethylene oxide, a block copolymer derived from the addition of ethylene oxide and propylene oxide to ethylenediamine, polyethelene glycol, or polyethylene oxide.

16. The composition of claim 14, wherein the surfactant is polyoxyethylene sorbitan monolaureate, polyoxyethylene sorbitan monooleat, or a block copolymer of propylene oxide and ethylene oxide is of a formula HO-(ethylene oxide)ₓ-(propylene oxide)_{y}-(ethylene oxide)ₓ—H, wherein x is about 79, y is about 28, and x′ is about 79.

17. The composition of claim 14, wherein the surfactant is present in a range from about 0.1 wt % to about 5 wt % of the composition.

18. A pharmaceutical composition, comprising the composition of claim 1 and a pharmaceutically acceptable excipient.

19. The pharmaceutical composition of claim 18, wherein the composition is an immediate release formulation.

20. The pharmaceutical composition of claim 18, wherein the composition is fluidly injectable at 25° C.

21. A dosage kit comprising the composition of claim 1 and a syringe.

22. The dosage kit of claim 21, wherein the syringe is divided such that the biologically active agent and the vehicle are separate until being mixed before injection.

23. A vehicle for combining with a biologically active agent to form a suspension composition, the vehicle comprising:
a hydrophilic viscosity enhancer; and
a solvent.

24. The vehicle of claim 23, further comprising a surfactant.

25. A method of administering a biologically active agent, comprising:
suspending the biologically active agent in the composition of claim 23; and
injecting the resulting composition into a patient in need thereof.

26. A method of making an injectable formulation of biologically active agent in a concentration of at least 50 mg/mL, comprising:
suspending the biologically active agent in the composition of claim 23.
